(19) United States
(12) Patent Application Publication
(10) Pub. No.: US 2025/0388526 A1
(43) Pub. Date: Dec. 25, 2025

(54) PROCESS FOR PRODUCING HYDROFLUOROETHERS

(71) Applicant: SOLVAY SPECIALTY POLYMERS ITALY S.P.A., Bollate (Milano) (IT)

(72) Inventors: Emanuela Antenucci, Saronno (IT); Letanzio Bragante, DUE CARRARE (PD) (IT); Manuel Gregori, Rovellasca (IT); Stefano Millefanti, Tradate (IT)

(73) Assignee: SOLVAY SPECIALTY POLYMERS ITALY S.P.A., Bollate (Milano) (IT)

(21) Appl. No.: 18/879,654

(22) PCT Filed: Jun. 22, 2023
(86) PCT No.: PCT/EP2023/066985

§ 371 Date: Dec. 27, 2024

(30) Foreign Application Priority Data
Jun. 29, 2022 (EP) 22181821.4

(51) Int. Cl. C07C 41/14; C07C 41/42

(52) U.S. Cl. CPC C07C 41/14; C07C 41/42

(57) ABSTRACT

A process for making hydrofluoroethers, the process comprising: •A) providing a mixture comprising one more polar aprotic organic solvents having a boiling point measured at 1 atm (1.01 bar) of from 60° C. to 170° C. and one or more chemical compounds carrying at least one —OH group which is part of an alcohol or of a phenol group, and •B): reacting said one or more chemical compounds carrying at least one —OH group with one or more fluorinated olefin in the presence of a basic catalyst thus providing a reacted mixture comprising one or more hydrofluoroethers.

### TECHNICAL FIELD

This application claims priority from the European patent application filed on 29 Jun. 2022 in EUROPE with Nr 22181821.4, the whole content of this application being incorporated herein by reference for all purposes.

The invention relates to a process for preparing hydrofluoroethers starting from mono or poly functional alcohols or phenols and fluorinated olefins.

### BACKGROUND ART

Various methods for producing hydrofluoroethers are known in the art. U.S. Pat. No. 4,208,081 (Du Pont) describes the preparation of hydrofluoroethers using ethylene glycol in a diethyl ether solution and tetrafluoroethylene (TFE). However the reaction is not complete as two reaction products are formed in almost equal amounts, one where both —OH groups of the ethylene glycol are converted to ether groups, and another where only one of the —OH groups of the ethylene glycol has been converted to ether, while the other remains as a free hydroxyl group.

RU1810324 to Natalya Guseva reports the preparation of hydrofluoroethers from a reaction between ethylene glycol and TFE in diglyme solvent in a anhydrous process with a 78% yield.

While processes to produce hydrofluoroethers are known, there is still a need for processes having a high yield, namely a better yield than prior art processes.

### SUMMARY OF THE INVENTION

The present invention relates to a process for making hydrofluoroethers, the process comprising:
A) providing a mixture comprising one more polar aprotic organic solvents having a boiling point measured at 1 atm of from 60° C. to 170° C. and one or more chemical compounds carrying at least one —OH group which is part of an alcohol or of a phenol group,
B): reacting said one or more chemical compounds carrying at least one —OH group with one or more fluorinated olefin in the presence of a basic catalyst thus providing a reacted mixture comprising one or more hydrofluoroethers.

### DETAILED DESCRIPTION OF THE INVENTION

It is thus an object of the present invention to provide a process for the preparation of hydrofluoroethers deriving from the reaction between a chemical compound carrying at least one —OH group which is part of an alcohol or of a phenol group with a fluorinated olefin (including partially and fully fluorinated olefins), which advantageously provides high yields and can be performed easily in mild conditions and without requiring ingredients which are harmful for the environment.

Within the context of the present invention a “hydrofluoroether” is defined as a chemical compound having the general formula R—O—R′ wherein at least one of R and R′ comprises at least one C—F bond and at least one C—H bond.

One way of forming hydrofluoroethers is to react a chemical compound carrying at least one —OH group which is part of an alcohol or of a phenol group with a fluorinated olefin which can be partially or fully fluorinated. The reaction between the —OH group and the C═C double bond of the olefin can be described as an addition to the C═C double bond where one of its carbon atoms forms a C—O bond and the other a C—H bond.

In a first step (A) of the process of the present invention a mixture is provided comprising one or more polar aprotic organic solvents and one or more chemical compounds carrying at least one —OH group which is part of an alcohol or of a phenol group.

Suitable polar aprotic organic solvents for use in the process of the present invention are polar aprotic organic solvents having a boiling point measured at atmospheric pressure (1 atm) of from of 60 to 170° C., preferably of from 70° C. to 90° C.

Particularly suitable polar aprotic solvents for use herein are those carrying a nitrile group, a particularly preferred solvent is acetonitrile.

The other essential component of the mixture to be provided in step A of the process of the present invention is one or more chemical compound carrying at least one —OH group which is part of an alcohol or of a phenol group. In the present invention it is intended that an —OH group is part of an alcohol group when it is covalently bonded to an aliphatic carbon atom which is not part of a carbonyl group (C═O), and that an —OH group is part of a phenol group when it is covalently bonded to an aromatic carbon (i.e. a carbon which is part of an aromatic ring).

These chemical compounds typically correspond to the general formula R₁—OH wherein R₁ can be any radical provided that the oxygen atom in the —OH group is covalently bonded to R₁ either to an aliphatic carbon atom, not also part of a carbonyl group as described above, or to an aromatic carbon atom.

Aside from this requirement R₁ is not particularly limited and can for example be selected from an aliphatic carbon radical which can be linear, branched and/or comprise cyclic moieties, an aromatic carbon radical which aromatic ring may or may not have other substituents and an aliphatic carbon radical which comprises one or more aromatic rings along the chain (for aliphatic carbon radical it is meant a radical where the radical centre is on an aliphatic carbon, for aromatic carbon radical it is meant a radical where the radical centre is on an aromatic carbon).

R₁ may also include other functional groups and hetero atoms, in particular it can comprise oxygen hetero atoms, preferably as part of other alcohol or phenol groups or engaged in ether bonds. R₁, in the case wherein the —OH containing compound of the invention is a polyfunctional alcohols or phenols, includes additional —OH groups. Examples of chemical compound carrying at least one —OH group suitable in the present invention are methanol, ethanol, n-propanol, iso-propanol, cyclohexanemethanol, cyclohexanol, ethylene glycol, di-ethylene glycol, tri-ethylene glycol, propylene glycol, di-propylene glycol, tri-propylene glycol, 1,3-propan-diol, penthaerythritol, cyclohexandiol, cyclohexanedimethanol, allyl alcohol, phenol, substituted phenol such as cresol, methoxyphenol, fluorophenol, chlorophenol, benzene diols (such as resorcinol, catechol, hydroquinone) and triols.

The one or more chemical compound carrying at least one —OH group for use in the present invention can be non halogenated, partially halogenated or fully halogenated, in case it is halogenated the halogens can be preferably selected from Cl and F.

While essentially any chemical compound carrying at least one —OH group which is part of an alcohol or of a phenol group as defined above can be used in the process of the present invention, the applicant has surprisingly found that the process of the invention is very effective when the chemical compound carrying at least one —OH group is a polyfunctional alcohol and in particular a bifunctional alcohol. In fact when forming an hydrofluoroether from a polyfunctional alcohol the yields tend to be lower as the various —OH groups may have different reactivity, especially after one or more of them have already reacted with the fluorinated olefin to form a first ether bond. The method of the invention allows to obtain an excellent yield also with polyfunctional alcohols. Preferred polyfunctional alcohols for the present invention are ethylene glycol, di-ethylene glycol, tri-ethylene glycol, propylene glycol, di-propylene glycol, tri-propylene glycol. A particularly preferred polyfunctional alcohol for the present invention is ethylene glycol.

Also the present invention is very effective when the chemical compound carrying at least one —OH group is selected from phenols and benzene diols and triols. The method of the invention produces a very good yield also with these types of —OH containing molecules. Preferred phenols for use herein are phenol, cresol, methoxyphenol, fluorophenol and chlorophenol.

In step A of the process of the present invention one or more chemical compound carrying at least one —OH group as defined above is provided in a mixture with one or more polar aprotic solvents selected as defined above. Such solvents are generally good solvents for the —OH carrying compounds so that preferably the mixture provided is homogeneous. The relative amount of the selected one or more polar aprotic organic solvents, and of the one or more chemical compounds carrying at least one —OH groups is preferably at least 1:1 by weight, preferably at least 2:1 more preferably at least 3:1, most preferably at least 4:1. Other solvents may be present in the mixture, but preferably the total amount of the one or more —OH carrying compounds and of the one or more selected polar aprotic solvents, represents at least 70%, more preferably at least 80%, even more preferably at least 90%, most preferably at least 95% by weight of the mixture.

The mixture provided in step A is reacted in step B of the process of the invention with one or more fluorinated olefin. Any fluorinated olefin can be used in the present invention, these include fully fluorinated olefins, and partially fluorinated olefins. Partially fluorinated olefins includes olefins which are fully halogenated (i.e. where all hydrogens are replaced with halogens provided that at least one hydrogen is replaced with a fluorine atom, for example TFE: tetrafluoroethylene, hexafluoropropylene: HFP CTFE: chlorotrifluoroethylene, perfluoromethylvinylether: PMVE, perfluoroethylvinylether: PEVE, or perfluoropropylvinylether: PMVE) and those which are partially halogenated so that they contain at least one C—F bond and at least one C—H bond and may contain also one or more bonds between carbon and an halogen different from F such as Cl, Br or I, typically Cl (for example vinylidene fluoride: VDF, trifluoroethylene: TrFE). Fully halogenated olefins are preferred, more preferred are fully fluorinated olefins.

In one embodiment fluorinated olefins for use in the process of the invention are those represented by the following formula:

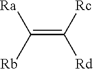

wherein Rₐ, R_{b}, R_{c} and R_{d} are each independently selected from the group consisting of H, F, Cl and hydrocarbon groups, possibly comprising one or more chlorine and/or fluorine atoms, optionally having one or more heteroatoms different from F and Cl, e.g. oxygen, possibly directly linked to the double bond, with the proviso that the fluorinated olefin comprises at least one C—F bond and that at least one of Rₐ, R_{b}, R_{c} and R_{d} is selected from fluorine or chlorine.

Preferably, Rₐ, R_{b}, R_{c} and R_{d} are each independently selected in the group consisting of F, Cl, C₁-C₄ perfluorocarbon groups, C₁-C₄ oxygen-containing perfluorocarbon groups, C₁-C₄ fluorochlorohydrocarbon groups, and C₁-C₄ oxygen-containing fluorochlorohydrocarbon groups. More preferably, at least three of Rₐ, R_{b}, R_{c} and R_{d} are selected from F, Cl and mixtures thereof.

As examples of such fluorinated olefins, mention may be made of tetrafluoroethylene (TFE), hexafluoropropylene (HFP), octafluorobutene, perfluoropentene, perfluorohexene, perfluoroheptene, perfluorooctene, perfluorocyclobutene, perfluorocyclopentene, perfluorocyclohexene, chlorotrifluoroethylene, dichlorodifluoroethylene, chloropentafluoropropene, perfluorobutadiene, perfluoromethylvinylether, perfluoroethylvinylether, perfluoropropylvinylether; CF₃OCCl═CClF, trichloroethylene, tetrachloroethylene, dichloroethylene isomers; and fluorodioxoles of formula:

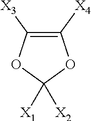

wherein X₁, X₂, X₃, and X₄, equal to or different from each other, are independently selected from F, R_{f} and OR_{f}, wherein R_{f} is a (per) fluorocarbon group, and wherein at least one of X₃, and X₄ is fluorine. Preferably the fluorinated olefin is selected among the fully halogenated olefins and more preferably from the group consisting of perfluoromethyl vinyl ether, perfluoroethyl vinyl ether, perfluoropropyl vinyl ether, tetrafluoroethylene (TFE), chlorotrifluoroethylene (CTFE), hexafluoropropylene (HFP) and more preferably is TFE.

The fluorinated olefin can be initially loaded in the reaction vessel or can be advantageously continuously fed in the required amount during the reaction.

In step B of the process of the present invention the mixture provided in step A is reacted with a fluorinated olefin in the presence of a basic catalyst.

The reaction of an —OH carrying compound with a fluorinated olefin can be schematized as follows:

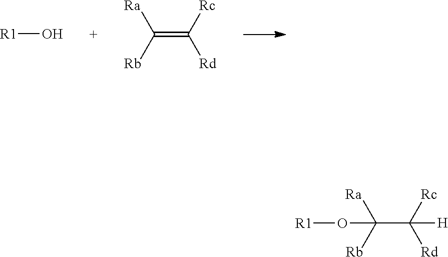

To note the radical R₁, in accordance with the definition provided above may still contain other —OH groups (e.g. in case of a polyfunctional alcohol). In that case also the resulting hydrofluoroether will still contain the same —OH groups so that it can further react with other molecules of the fluorinated olefin until all the —OH groups are fully reacted so that their oxygen atoms are all engaged in ether type bonds.

The reaction can be typically performed in a stirred reactor which is preferably sealed. The molar ratio between the —OH carrying compounds and the fluorinated olefins is in principle stoichiometric i.e. in order to have complete reaction and not have residual reagents the same molar amount of double bonds from the olefins should be present as the molar amount of —OH groups. Naturally in the case of polyfunctional alcohols each molecule carrying more than one —OH can react with multiple molecules of fluorinated olefin (one for each —OH group) so that for example one mole of ethylene glycol will stoichiometrically react with two moles of olefin. While a molar ratio 1:1 between double bonds and OH groups is ideal, the present invention can be effectively carried out also when one of the components is in a molar excess up to 50%, preferably up to 30%, more preferably up to 20% most preferably up to 10%. In case one component is in excess said component in excess is preferably the fluorinated olefin.

The basic catalyst can be any chemical compound capable of creating a basic environment i.e. to subtract protons from the reagents thus promoting the ionic reaction. Preferred basic catalyst are selected from inorganic hydroxides (such as NaOH, KOH, LiOH, Ca(OH)2, Mg(OH)2), inorganic salts of weak acids (such as alkali metal phosphates or carbonates), organic basic compounds (such as alcolates). Most preferred basic catalysts are NaOH and KOH.

The amount of catalyst to be used is typically from 5% to 100%, preferably 10%-70%, more preferably 15%-50% by moles with respect to the total moles of —OH groups.

Typically the basic catalyst is added to the reactor adding it to the mixture provided in step A under agitation. The reactor is then typically sealed and the fluorinated olefin is pumped in gas form up to a pressure of from 1 to 50 bar, preferably 2-30 bar, more preferably 3-20 bar, most preferably between 4 and 14 bar. In case the fluorinated olefin is in liquid form the olefin can be introduced as a liquid and if it remains in liquid status at the temperature of reaction, the reaction can be carried out at a lower pressure or even at atmospheric pressure.

The reaction typically starts immediately. Preferably, during the reaction, the reactor is maintained at a temperature of from 20° to 90° C., preferably from 30° to 80° C., most preferably from 40° to 70° C. The reaction time can be variable depending on the temperature, pressure and reagents used. Typically the reaction will require from 1 to 20 hours to complete.

After the reaction is completed the reactor is typically vented to remove the excess of fluorinated olefin. At this stage the reactor contains a liquid reacted mixture comprising one or more hydrofluoroethers and the one or more organic solvents along with residues of the basic catalyst and small amounts of reaction by-products.

The hydrofluoroethers can be extracted directly from the reacted mixture with known techniques such as distillation in optional step C of the present invention. However the reacted mixture obtained in step B still contains dissolved or dispersed solids, typically inorganic solids deriving from the basic catalyst, so that the direct distillation of said reacted mixture would cause the build-up of unwanted solid deposits on the distillation equipment which, while it can be acceptable in lab scale, are more problematic at an industrial scale as it could force the equipment to have frequent stops for cleaning/restoring it. Therefore, preferably, before extracting the hydrofluoroethers via distillation the reacted mixture is purified to remove catalyst residues and solid by-products.

In one embodiment the reacted mixture is purified via an extraction with water. In another embodiment the reacted mixture is purified trough evaporation and re-condensation. These two embodiments will be described in detail below.

### Evaporation/Recondensation Method:

In the optional step D of the present invention the liquid reacted mixture directly resulting from the reaction of step B is completely evaporated and recondensed in liquid form thereby obtaining a purified reacted mixture. Any available technique can be used to evaporate the liquid reacted mixture, for example heating and a vacuum can be used individually or in combination to evaporate the mixture. A conventional evaporation equipment (e.g. a rotary evaporation equipment) may be used. Following the evaporation of the liquid reacted mixture, a solid residue is formed comprising the residual basic catalyst and salts obtained as by-products of the reaction which can be discarded or recycled.

The purified reacted mixture obtained in step D, differently from the reacted mixture obtained in step B, is pure enough to be distilled in a conventional distillation equipment. This is performed in step E of the process of the present invention.

### Water Extraction Method:

In the optional step F of the process of the present invention the liquid reacted mixture directly resulting from the reaction of step B is mixed with water and subject to agitation and or stirring so to extract in the water phase the water soluble impurities such as residues of the basic catalyst and other impurities and by-products. The relative amounts of water and reacted mixture to use in this step are from 1:15 to 15:1 by weight, preferably from 1:5 to 5:1, more preferably from 2:1 to 1:2. Agitation can be performed with any suitable technique used for extractions as known to the skilled person and a separatory funnel or similar equipment can be used to separate the water phase from the phase containing the aprotic polar solvent and the hydrofluoroether. The resulting phase containing the aprotic polar solvent and the hydrofluoroethers once separated from the water phase constitutes the purified reacted mixture which is pure enough to be subject to distillation in a conventional distillation equipment in step G of the process of the present invention.

In all embodiments distillation allows to separate the hydrofluoroethers from the solvent and if necessary among themselves (in case more than one hydrofluoroethers is obtained). Distillation can be performed using conventional techniques and if necessary can be repeated to further purify the individual components. In general the solvent will be recovered with known methods in order to be reused.

The Evaporation/recondensation method described above is in general preferred to the water extraction method because the water extraction method generates a large amount of waste water which is contaminated with the impurities of the system and therefore needs to be treated before being discarded or reused.

The process of the invention can be carried out under mild conditions, additionally and a very high yield of hydrofluoroethers is obtained.

The invention will be now described in more detail with reference to the following examples, whose purpose is merely illustrative and not intended to limit the scope of the invention. Should the disclosure of any patents, patent applications, and publications which are incorporated herein by reference conflict with the description of the present application to the extent that it may render a term unclear, the present description shall take precedence.

### EXAMPLES

Product identification were performed by NMR (F-NMR and H-NMR) and GC and GC-MS analysis (GC using CP-WAX52CB column and CP-Sil8CB column for GC-MS peaks attribution).

### Example 1-Ethylene Glycol+TFE+Evaporation

37 g of ethylene glycol, 211 g of acetonitrile and 9.4 g of sodium hydroxide were loaded into a 600 ml stirred Hastelloy reactor. After purging with nitrogen and vacuum at 0.3 bar, the reactor was heated at 50° C. and, under stirring, pressurized up to 11 bar with TFE (tetrafluoroethylene). After 6 hours stirring was stopped, reactor was cooled and after purging residues of TFE with nitrogen, a reacted mixture was recovered rinsing with 106 g of additional acetonitrile and discharged.

The collected reacted mixture (480 g) was then transferred in a glass flask and evaporated under vacuum at 1 mbar in a rotary evaporator heating the flask at 90° C. Solid by-products (14.5 g) were discarded and a clear colorless purified reacted mixture (445 g) was collected.

The purified reacted mixture was then distilled in a glass distillation equipment with a flask, a packed column and a condenser, equipped with vacuum pump. Distillation was carried out increasing temperature from 100° C. to 155° C. and with reduced pressure from 950 mbar down to 50 mbar. The distilled product contained 142 g of diether HCF₂CF₂—O—CH₂CH₂—O—CF₂CF₂H and 0.02 g of monoether HCF₂CF₂—O—CH₂CH₂—OH (0.01% by weight of the diether). Yield of the overall process based on the load of ethylene glycol was 90.3%.

### Example 2-Ethylene Glycol+TFE+Water Extraction

7231 g of acetonitrile, 2200 g of ethylene glycol and 1290 g of sodium hydroxide were loaded into a 22 L stirred Hastelloy reactor. After purging 4 times with nitrogen and vacuum at 0.3 bar, the reactor was heated at 60° C. and, under stirring, pressurized up to 3.5 bar with TFE (tetrafluoroethylene) and TFE was continuously fed maintain the initial pressure. During the reaction the reactor was cooled since the reaction was vigorous and exothermic. About 1300 g/h of TFE were consumed. After 7.5 hours the reaction stopped and TFE supply was suspended. The reactor was left at 60° C. and stirring and cooled to 25° C., excess TFE was purged with nitrogen, a reacted mixture was recovered and discharged. The collected reacted mixture was then transferred in 200 L vessel and washed with 150 L demineralized water stirring at 25° C. for 6 hours and left to separate for 10 hours. The bottom organic layer was then separated (9300 g) and batch distilled in a rectification column (length 2.70 m, internal diameter 40 mm, filled with packing ¼″ Teflon® cylinders, and a 10 L bottom reboiler).

Distillation of the hydrofluoroethers was carried out under reduced pressure (80 mbar at 81° C. head reflux ratio 10:1).

The hydrofluoroether mixture obtained was 8060 g, and was found to contain 8052 g of diether HCF₂CF₂—O—CH₂CH₂—O—CF₂CF₂H and 8 g of monoether HCF₂CF₂—O—CH₂CH₂—OH (0.1% by weight of the diether). Yield of the overall process based on the load of ethylene glycol was 86%.

### Example 3-Phenol+TFE+Water Extraction

3000 g of phenol, 3956 g of acetonitrile and 420 g of sodium hydroxide were loaded into a 22 L stirred Hastelloy reactor. After purging 4 times with nitrogen and vacuum at 0.3 bar, the reactor was heated at 60° C. and, under stirring, pressurized up to 3.8 bar with TFE (tetrafluoroethylene) and TFE was continuously fed to maintain the initial pressure. When the reaction started the temperature increased due to exothermic reaction, the temperature of the reactor was controlled at 70° C. for 8.5 hours (total TFE fed was 3536 g). After cooling the reactor was purged with nitrogen, a reacted mixture was recovered and discharged. The reacted mixture was then washed with 3 times its volume of a water phase containing demineralized water and 4% by weight of sodium chloride. The organic layer was then separated as a purified reacted mixture. The purified reacted mixture was then distilled in a 70 cm rashig i.d. column 30 mm with reflux head condenser. The distillation product was a composition comprising the hydrofluoroether (5999 g) and the vinylether C₆H₅—O—CF═CF₂ (3.7 g, 0.06% by weight of the hydrofluoroether.

The overall yield was 96.4% based on the phenol.

When comparing the purity and the yield of the process of the present invention with that of the prior art it is clear that the process of the present invention provides for higher yields, higher purity of the hydrofluoroethers obtained.

Possible modifications and/or additions may be made by those skilled in the art to the hereinabove disclosed and illustrated embodiment while remaining within the scope of the following claims.

Should the disclosure of any patents, patent applications, and publications which are incorporated herein by reference conflict with the description of the present application to the extent that it may render a term unclear, the present description shall take precedence.

1. A process for making hydrofluoroethers, the process comprising:
A) providing a mixture comprising one or more polar aprotic organic solvents having a boiling point measured at 1 atm of from 60° C. to 170° C. and one or more chemical compounds carrying at least one —OH group which is part of an alcohol or of a phenol group, and
B) reacting said one or more chemical compounds carrying at least one —OH group with one or more fluorinated olefins in the presence of a basic catalyst thus providing a reacted mixture comprising one or more hydrofluoroethers.

2. The process according to claim 1 wherein said fluorinated olefin is a fully halogenated olefin.

3. The process according to claim 2 wherein said fluorinated olefin is selected from the group consisting of perfluoromethyl vinyl ether, perfluoroethyl vinyl ether, perfluoropropyl vinyl ether, tetrafluoroethylene, hexafluoropropylene, and chlorotrifluoroethylene.

4. The process according to claim 3 wherein said fluorinated olefin is tetrafluoroethylene.

5. The process according to claim 1 wherein said one or more chemical compounds carrying at least one —OH group are polyfunctional alcohols.

6. The process according to claim 5 wherein said one or more chemical compounds carrying at least one —OH group is selected from the group consisting of ethylene glycol, di-ethylene glycol, tri-ethylene glycol, propylene glycol, di-propylene glycol, tri-propylene glycol and mixtures thereof.

7. The process according to claim 5 wherein said one or more chemical compounds carrying at least one —OH group is ethylene glycol.

8. The process according to claim 1 wherein said one or more chemical compounds carrying at least one —OH group are phenols.

9. The process according to claim 8 wherein said one or more chemical compounds carrying at least one —OH group is selected from the group consisting of phenol, cresol, methoxyphenol, fluorophenol and chlorophenol.

10. The process according to claim 1 wherein said one or more polar aprotic organic solvent is acetonitrile,

11. The process according to claim 1, comprising an additional step C wherein said hydrofluoroethers are directly extracted from said reacted mixture.

12. The process according to claim 1 wherein said process further comprises the steps of
D) evaporating completely said reacted mixture and re-condensing it in liquid form as a purified reacted mixture, and
E) separating said hydrofluoroethers in purified form from said purified reacted mixture via distillation.

13. The process according to claim 1 wherein said process further comprises the steps of
F) mixing said reacted mixture with water, agitating the mixture thereby extracting water soluble impurities from said reacted mixture, and separating the purified reacted mixture as a water immiscible phase, and
G) separating hydrofluoroethers in purified form from said purified reacted mixture via distillation.

14. The process of claim 11, wherein the direct extraction is via distillation.
